(19) United States
(12) Patent Application Publication
(10) Pub. No.: US 2016/0016002 A1
(43) Pub. Date: Jan. 21, 2016

(54) Treatment of Diseases Using Combination of Ultraviolet Blood Irradiation and Probiotics

(71) Applicant: John Todd Kuenstner, Charleston, WV (US)

(72) Inventor: John Todd Kuenstner, Charleston, WV (US)

(21) Appl. No.: 14/802,442

(22) Filed: Jul. 17, 2015

Related U.S. Application Data
(60) Provisional application No. 62/025,586, filed on Jul. 17, 2014.

(51) Int. Cl. A61N 5/06; A61K 35/741

(52) U.S. Cl. CPC A61N 5/0613; A61K 35/741; A61N 2005/0661

(57) ABSTRACT

A method of and composition for treating a patient having Mycobacterium avium complex (MAC), and in embodiments including Mycobacterium avium subspecies paratuberculosis (MAP) and/or Mycobacterium avium subspecies hominissuis (MAH), causing one or more diseases, one embodiment of the method including administering to the patient an effective amount of one or more probiotics and administering to the patient an effective amount of ultraviolet blood irradiation (UVBI) treatment, and an embodiment of the composition including an effective amount of one or more probiotics and an effective amount of UVBI treated blood of the patient.

### CROSS REFERENCE TO RELATED APPLICATION

This application claims priority to U.S. provisional patent application No. 62/025,586 filed Jul. 17, 2014, which is incorporated herein in by reference in its entirety.

### FIELD OF THE INVENTION

This invention is related to the treatment of diseases, and more particularly to the treatment of Mycobacterium avium complex (MAC), and in embodiments Mycobacterium avium subspecies paratuberculosis (MAP) and/or Mycobacterium avium subspecies hominissuis (MAH), causing one or more diseases using a combination of probiotics and/or ultraviolet blood irradiation (UVBI).

### BACKGROUND OF THE INVENTION

Mycobacterium avium subspecies paratuberculosis (MAP) is a bacterium that causes Johne's disease, a chronic diarrheal wasting disease in cattle and a chronic wasting disease in sheep and goats. This bacterium has long been suspected to cause Crohn's disease. A diarrheal/wasting illness associated with infection with MAP has also been reported in non-human primates. The viable bacterium is found in commercially available pasteurized milk. In fact, 2.7% of retail pasteurized milk samples purchased in Wisconsin, Minnesota and California were found to contain viable MAP. Because of the prevalence of this organism in the food chain and because Johne's disease is a worldwide zoonosis, it should not be surprising that the first mass screening of the human population in a study done in North India on 26,390 serum samples submitted for multiple medical conditions including diabetes, liver disorders, anemia, thyroid, tuberculosis, typhoid, abdominal disorders, inflammatory illness and ion imbalance, showed that 34% of the samples had evidence of MAP infection by an ELISA antibody test. The same study showed that 12.7% of apparently normal individuals had IS900 PCR evidence of MAP in their blood.

Once an animal is infected, the MAP bacterium grows and multiplies inside the cells of the immune system. The organism is excreted in the feces, and to a lesser extent in milk. Outside the host animal, the bacterium multiplies poorly, but can survive over a year in the environment because of its resistance to heat, cold and the effect of drying. This slow-growing bacterium affects the ileum and causes diarrhea and cachexia. There is no known curative treatment for Johne's disease.

It has been suggested that there may be an association between Crohn's disease (CD) and Johne's disease. Studies have shown an increase in the detection and isolation of MAP in adult Crohn's patients and in children newly diagnosed with Crohn's disease, and that most patients with Crohn's disease may have MAP. Crohn's disease, like Johne's disease, has been treated with antibiotic therapy. However, patients undergoing such treatment for Crohn's disease may have a significant relapse rate and thus such treatments may not be optimal. Favorable results in the treatment of Crohn's disease using conventional therapies may be only around 30 percent.

Therefore, there may be a need for a different and/or more expanded therapy that may be more effective than conventional methods in fighting Crohn's disease and other autoimmune diseases.

### SUMMARY OF THE INVENTION

In an embodiment, a method of treating a patient having Mycobacterium avium complex (MAC) causing one or more diseases includes: administering to the patient an effective amount of one or more probiotics; and administering to the patient an effective amount of ultraviolet blood irradiation (UVBI) treatment.

In another embodiment, a composition for treating a patient having Mycobacterium avium complex (MAC) causing one or more diseases includes: an effective amount of one or more probiotics; and an effective amount of ultraviolet blood irradiation (UVBI) treated blood of the patient.

In another embodiment, a method of treating a patient having Mycobacterium avium subspecies paratuberculosis (MAP) causing one or more autoimmune diseases includes: administering to the patient an effective amount of one or more probiotics; and administering to the patient an effective amount of ultraviolet blood irradiation (UVBI) treatment.

### DETAILED DESCRIPTION

Reference will now be made to embodiments of compositions and methods for treating Mycobacterium avium complex (MAC) such as, for example, species M avium or M intracellulare (also collectively previously referred to as Mycobacterium avium-intracellulare (MAI)), and in embodiments including Mycobacterium avium subspecies paratuberculosis (MAP) and/or Mycobacterium avium subspecies hominissuis (MAH), causing one or more diseases. Details, features, and advantages of the treatment compositions and methods will become further apparent in the following detailed description of embodiments thereof.

Any reference in the specification to “one embodiment,” “a certain embodiment,” or a similar reference to an embodiment is intended to indicate that a particular feature, structure or characteristic described in connection with the embodiment is included in at least one embodiment of the invention. The appearances of such terms in various places in the specification do not necessarily all refer to the same embodiment.

References to “or” are furthermore intended as inclusive, so “or” may indicate one or another of the ored terms or more than one ored term.

As used herein, the term “effective amount” refers to that amount of a substance desired to bring about a desired effect in a patient. Regarding autoimmune diseases as described herein, the “effective amount” refers to the amount of a substance to treat one or more of the autoimmune diseases and MAC, and in embodiments specifically MAP and/or MAH. As used herein, the term “patient” refers to any member of the animal kingdom, including but not limited to animals and homo sapiens.

Accordingly, the present invention includes a method and composition for treating a patient having MAC, and in embodiments including MAP and/or MAH, causing one or more autoimmune diseases comprising administering to the patient ultraviolet blood irradiation (UVBI) and at least one probiotic. In an embodiment, the one or more probiotics includes a bacterial probiotic. For example, in an embodiment, the one or more probiotics includes Dietzia. The one or more probiotics may be administered orally. The treatment may be periodic. Other embodiments of the present invention include a composition for treating a patient having MAC, and in embodiments including MAP and/or MAH, causing one or more autoimmune diseases comprising UVBI treated blood and at least one probiotic such as, in various embodiments, a bacterial probiotic such as Dietzia and/or another probiotic or probiotics. The composition and method may treat an underlying infection by MAC, and in embodiments specifically including MAP and/or MAH, causing the one or more autoimmune diseases.

In an embodiment, one or more antibiotics may also be administered in addition to the UVBI and Dietzia or other probiotic. The one or more antibiotics may be, for example, in various embodiments, one or more of clarithromycin, azithromycin, rifampicin, rifabutin, clofazimine, ciprofloxacin, ethambutol, and metronidazole. The method and composition may treat an underlying infection by MAC, including in embodiments specifically MAP and/or MAH. One or more alternative or additional antibiotics may be used.

In an embodiment in which one or more antibiotics is administered in addition to the UVBI and Dietzia and/or another probiotic or probiotics, the Dietzia and/or other probiotic(s) may be administered after a period of administration of UVBI and one or more antibiotics. For example, in an embodiment, Dietzia may be administered to the patient after about three months of treating the patient with UVBI and the one or more antibiotics. The Dietzia treatment may begin at another time.

In one embodiment, Vitamin A and/or Vitamin D is added to the combination therapy of UVBI and one or more probiotics such as Dietzia and possibly including one or more of the aforementioned or other antibiotics.

The UVBI treatment in embodiments of the method of treating a patient having MAC, and in embodiments including MAP and/or MAH, causing one or more autoimmune diseases may include any method of providing UVBI. For example, the UVBI treatment may include inserting a catheter into a vein of a forearm of a patient, removing 200 cc or another amount of blood from the patient, irradiating the removed blood using ultraviolet light, and then returning that blood to the bloodstream of the patient. The irradiation of the blood with ultraviolet light may be by way of passing the blood through a quartz cuvette or other tubing where it is exposed to ultraviolet light. Other UVBI methods and devices may be used in other embodiments. The UVBI treated blood in embodiments of the compositions for treating a patient having MAC, and in embodiments including MAP and/or MAH, causing one or more autoimmune diseases may include UVBI treated blood of the patient from the aforementioned or another UVBI treatment method and/or device.

The one or more autoimmune diseases may include, but are not limited to, one or more of Crohn's disease, ulcerative colitis, type 1 diabetes mellitus, multiple sclerosis, complex regional pain syndrome, hypothyroidism, idiopathic thrombocytopenic purpura, lymphangiomatosis, sarcoidosis, Sjogren's disease, myasthenia gravis, scleroderma, systemic lupus erythematosus, psoriasis, ankylosing spondylitis, rheumatoid arthritis, systemic sclerosis, and fibromyalgia.

In various embodiments, the method of treating MAC, and in embodiments including MAP and/or MAH, causing one or more autoimmune diseases, such as those provided herein, comprises administering to a patient an effective amount of at least one probiotic and at least one UVBI treatment. For example, the treatment method may include the administration to a patient of at least 10″ viable cfu (colony forming units) per 100 pounds body weight per day of a probiotic. That probiotic may be a bacterial probiotic in an embodiment, and specifically Dietzia in an embodiment. The method may include, in one preferred embodiment, concurrently administering to the patient the one or more probiotics, the administration of the one or more probiotics also at least partially over the same time period as when the UVBI treatments are administered. The method may include administering to the patient UVBI treatment at least once a week for at least 12 weeks. In a preferred embodiment, the method includes starting the patient on the UVBI treatment from about two or more weeks earlier to the time the one or more probiotics begin to be administered to the patient. In a more preferred embodiment, the one or more probiotics are taken for at least two years and up to three years, though in other embodiments other durations of time may be used. In preferred embodiments, when multiple probiotics are taken, they may be taken concurrently at first and then cycled later, or they may be only taken concurrently, or they may only be cycled.

In various embodiments, the method may also include administering to the patient an effective amount of one or more antibiotics such as those provided above. For example, in an embodiment, the method may include administering to the patient at least 11 mg/kg/day of rifabutin or at least 9 mg/kg/day of rifampin. The method may further include administering to the patient at least 14 mg/kg/day of ciprofloxacin or at least 7 mg/kg/day of levofloxacin. In an embodiment, the method may include administering to the patient at least 4 mg/kg per week of clofazimine, such as when the patient is in clinical remission. As used herein, “clinical remission” is the partial or complete disappearance of the clinical and subjective characteristics of the one or more autoimmune diseases provided herein.

One embodiment of a therapy for MAP includes performing a blood culture of the patient's blood along with a culture or Polymerase Chain Reaction (PCR) test for MAP, and/or a MAP antibody test performed to confirm that any MAP causing one or more diseases, such as Crohn's disease in an embodiment, has been eradicated.

In various embodiments, a composition for treating a patient for MAC, and in embodiments including MAP and/or MAH, causing one or more autoimmune diseases, such as those provided herein, comprises an effective amount of at least one probiotic such as provided above and an effective amount of UVBI treated blood of the patient such as provided by the UVBI treatment method above. In one preferred embodiment, the one or more probiotics may be taken concurrently with respect to each other and also at least partially over the same time period as when the UVBI treated blood is returned to the bloodstream of the patient. In an embodiment, the composition further includes an effective amount of one or more antibiotics, and the composition may in an embodiment include Vitamin A and/or D such as described herein.

Vitamin D has been shown to play an important role in the host immune response to mycobacterial infection. Vitamins A and D have been shown to inhibit the growth of MAP in vitro. Vitamin D has also been shown to reduce the proliferation of M. tuberculosis in macrophages. Activated dendritic cells are known to produce Vitamin D and Vitamin D induces the intracellular production of cathelicidin, which is an antimicrobial protein. High levels of Vitamin D have been correlated with a reduced risk of developing multiple sclerosis, and Vitamin D intake is inversely associated with rheumatoid arthritis (another autoimmune condition) and the severity of this latter disease also correlates with Vitamin D levels. Finally, many types of cells including leukocytes and, in particular, monocytes, exposed to ultraviolet light secrete heat shock proteins and these proteins play an important role in the response to infection.

The combination of UVBI and Dietzia, possibly also with one or more antibiotics and possibly Vitamin A and/or D, may be synergistic in the treatment of autoimmune diseases targeting MAC, and in embodiments including MAP and/or MAH, as the combination may yield more beneficial results than would be predictable from the additive effects of each. The autoimmune disease conditions are expected to be lifelong, and yet complete resolution of these diseases symptomatically and from the standpoint of negative blood cultures and studies such as MAP antibody studies may occur from the combination of UVBI and Dietzia and possibly also one or more antibiotics and/or Vitamin A and/or D.

The treatment of the above diseases by the above methods is not heretofore known. Currently, none of the above “autoimmune diseases” is recognized by the medical community as an infectious disease. Rather, the theory is largely ridiculed by most physicians who are the thought leaders of gastroenterology and infectious disease.

While specific embodiments of the invention have been described in detail, it should be appreciated by those skilled in the art that various modifications and alternations could be developed in light of the overall teachings of the disclosure. Accordingly, the particular compositions and methods disclosed are meant to be illustrative only and not limiting as to the scope of the invention.

What is claimed is:

1. A method of treating a patient having Mycobacterium avium complex (MAC) causing one or more diseases, comprising:
administering to the patient an effective amount of one or more probiotics; and
administering to the patient an effective amount of ultraviolet blood irradiation (UVBI) treatment.

2. The method of claim 1, wherein the one or more probiotics comprise a bacterial probiotic.

3. The method of claim 1, wherein the one or more probiotics comprise Dietzia.

4. The method of claim 1, further comprising administering to a patient at least one of Vitamin A and Vitamin D.

5. The method of claim 1, further comprising administering to a patient an effective amount of at least one or more antibiotics.

6. The method of claim 5, wherein, the one or more antibiotics comprise one or more of clarithromycin, azithromycin, rifampicin, rifabutin, clofazimine, ciprofloxacin, ethambutol, and metronidazole.

7. A composition for treating a patient having Mycobacterium avium complex (MAC) causing one or more diseases, comprising:
an effective amount of one or more probiotics; and
an effective amount of ultraviolet blood irradiation (UVBI) treated blood of the patient.

8. The composition of claim 7, wherein the one or more probiotics comprise a bacterial probiotic.

9. The composition of claim 7, wherein the one or more probiotics comprise Dietzia.

10. The composition of claim 7, further comprising at least one of Vitamin A and Vitamin D.

11. The composition of claim 7, further comprising an effective amount of one or more antibiotics.

12. The composition of claim 11, wherein the one or more antibiotics comprise one or more of clarithromycin, azithromycin, rifampicin, rifabutin, clofazimine, ciprofloxacin, ethambutol, and metronidazole.

13. A method of treating a patient having Mycobacterium avium subspecies paratuberculosis (MAP) causing one or more autoimmune diseases, comprising:
administering to the patient an effective amount of one or more probiotics; and
administering to the patient an effective amount of ultraviolet blood irradiation (UVBI) treatment.

14. The method of claim 13, wherein the one or more probiotics comprise a bacterial probiotic.

15. The method of claim 13, wherein the one or more probiotics comprise Dietzia.

16. The method of claim 13, further comprising administering to the patient one or more antibiotics.

17. The method of claim 16, wherein the one or more antibiotics comprise one or more of clarithromycin, azithromycin, rifampicin, rifabutin, clofazimine, ciprofloxacin, ethambutol, and metronidazole.

18. The method of claim 13, further comprising administering to a patient at least one of Vitamin A and Vitamin D.

19. The method of claim 13, wherein the one or more autoimmune diseases comprise one or more of Crohn's disease, ulcerative colitis, type 1 diabetes mellitus, multiple sclerosis, complex regional pain syndrome, hypothyroidism, idiopathic thrombocytopenic purpura, lymphangiomatosis, sarcoidosis, Sjogren's disease, myasthenia gravis, scleroderma, systemic lupus erythematosis, psoriasis, and fibromyalgia.

20. The method of claim 13, further comprising performing at least one of a blood culture for MAP, a Polymerase Chain Reaction test for MAP, and a test for MAP antibodies to confirm that the MAP has been eradicated in the patient.
